(19) United States
(12) Patent Application Publication
(10) Pub. No.: US 2019/0077780 A1
(43) Pub. Date: Mar. 14, 2019

(54) Method for Preparing Halohydrin and Epoxide

(71) Applicant: Jiangxi Sukeer New Material Co., Ltd., Nanchang, Jiangxi (CN)

(72) Inventors: Xingtian Shu, Nanchang, Jiangxi (CN); Min Lin, Nanchang, Jiangxi (CN); Weilin Liao, Nanchang, Jiangxi (CN)

(21) Appl. No.: 16/085,054

(22) PCT Filed: Mar. 13, 2017
(86) PCT No.: PCT/CN2017/076389

§ 371 Date: Sep. 14, 2018

(30) Foreign Application Priority Data
Mar. 14, 2016 (CN) 201610143839.X

(51) Int. Cl. C07D 301/27; C07D 303/04; C07D 301/32; C07C 29/64; C07C 31/34

(52) U.S. Cl. CPC C07D 301/27; C07D 303/04; C07C 31/34; C07C 29/64; C07D 301/32

(57) ABSTRACT

Provided is a method for preparing an epoxide by halohydrination, the method comprising: (1) halohydrination: adding H₂O, a halogen(s) and an olefin compound to a reaction device for reaction to obtain a halohydrin; (2) saponification: saponificating the halohydrin with an alkali metal hydroxide to obtain an epoxide and an alkali metal halide; (3) performing a bipolar membrane electrodialysis of the alkali metal halide to obtain an alkali metal hydroxide and a halogen hydride. Also provided is a method for preparing an epoxide by halohydrination, the method comprising: (1) halohydrination: halohydrinating a halogen hydride, an H₂O₂ and an olefin compound to obtain a halohydrin; optionally, (2) saponification: saponificating the halohydrin with an alkali metal hydroxide to obtain an epoxide and an alkali metal halide; optionally, (3) performing a bipolar membrane electrodialysis of the alkali metal halide to obtain an alkali metal hydroxide and a halogen hydride. The method according to the present invention can prepare a halohydrin or an epoxide at very high selectivity and yield, and greatly reduce the amount of waste water and waste slag discharges.

### FIELD OF THE INVENTION

The invention relates to a method for preparing a halohydrin, and a method for preparing an epoxide from a halohydrin; and more specifically a method for preparing a halohydrin by halohydrination using an olefin as a raw material and a method for preparing an epoxy compound by halohydrination.

### BACKGROUND OF THE INVENTION

Epoxides contain epoxy groups, are chemically active, and are easy to be polymerized by ring-opening. They are important organic chemical raw materials. For example, propylene oxide is the third largest derivative of propylene, and its largest use is to prepare polyether polyols, and also to produce propylene glycol and nonionic surfactants. In recent years, PO has also been widely used to synthesize green products such as dimethyl carbonate and carbonate polymers.

The main industrialization methods for production of propylene oxide are chlorohydrination and co-oxidation. The co-oxidation method is further divided into ethylbenzene co-oxidation and isobutane co-oxidation. Most of China's enterprises use the chlorohydrin process, and also several sets of co-oxidation processes and hydrogen peroxide direct oxidation processes are put into use.

2-chloroethanol (also known as “chlorethyl alcohol”) is an important organic solvent and organic synthetic raw material. It is used in the manufacture of ethylene oxide, synthetic rubber, dyes, pharmaceuticals and pesticides, etc., and also used as an organic solvent. It is also used in the manufacture of ethylene glycol, ethylene oxide, and the synthesis of pharmaceuticals, dyes, pesticides and the like.

Ethylene oxide is an important organic chemical raw material. It is an organic compound of the chemical formula C₂H₄O, and a toxic carcinogen that was previously used to prepare bactericide. It is widely used in detergent, pharmaceutical, printing and dyeing industries. It can be used as a starter for detergents in chemical industry. Ethylene oxide (“EO”) is one of the simplest cyclic ethers and also is a heterocyclic compound. It is an important petrochemical product. Ethylene oxide is mainly used in the manufacture of ethylene glycol (polyester fiber raw materials), synthetic detergents, nonionic surfactants, antifreeze agents, emulsifiers and diglycol products, as well as plasticizers, lubricants, rubber and plastics, etc. it is widely used in dyeing, electronics, medicine, pesticides, textiles, paper-making, automotive, oil mining and refining and many other fields.

In the prior art, a method for preparing ethylene oxide comprises the following: 1) chlorohydrination in a two-step reaction, wherein the first step is to pass ethylene and chlorine into water to form 2-chloroethanol; and the second step is the reaction of 2-chloroethanol with a base (usually lime milk) to form ethylene oxide. 2) the oxidation method: it can be subdivided into air method and oxygen gas method. The former uses air as the oxidant, and the latter uses oxygen having a concentration greater than 95% by volume as the oxidant.

For the preparation of epoxides, its production by chlorohydrination has a long history. The main process of the chlorohydrination includes halohydrination of olefin, lime milk saponification and product refining. It is characterized by mature production process, higher operational load elasticity and good selectivity, low purity requirement on olefin raw materials, which can improve the safety of production and reduce construction investment. Due to the low investment in fixed assets and low product cost, the resulting products have strong cost competitiveness.

The chlorohydrination in prior art for preparing epoxide mainly has the following problems: (1) chlorine gas as raw material undergoes an side addition reaction with an olefin to form a dichloroalkane. Dichloropropane is difficult to use, resulting in waste of a lot of raw materials. In addition, the chlorine gas reacts with the olefin to release a large amount of heat, and the heat is concentrated in the reaction vessel, which causes a great danger in production. (2) The chlorine gas as raw material is inevitably entrained with a small amount of oxygen in the production process. As the reaction progresses, the chlorine gas continuously participates in the reaction. Therefore, it is necessary to continuously replenish chlorine gas, and oxygen cannot participate in the reaction, and is continuously accumulated in the reaction device. As the reaction proceeds, the oxygen concentration is continuously increased, and the reaction releases heat. Oxygen, chlorine, and olefins are present at the same time, and the explosion is likely to occur at high temperatures. (3) The reaction produces hydrochloric acid, and it is necessary to consume a large amount of a base (for example, calcium hydroxide) to neutralize the reaction product hydrochloric acid to facilitate the reaction. (4) The reaction produces a large amount of calcium chloride, which causes the produced wastewater to have a high COD and pollutes the environment. (5) The disadvantage of the chlorohydrination is that it consumes a large amount of water and produces a large amount of waste water and waste slag. In order to produce 1 ton of epoxide by chlorohydrination, the method will produces 40-50 tons of chloride-containing wastewater and 2.1 tons of calcium chloride residue. The wastewater has high temperature, high pH, high chloride content, high COD content and high suspended solid content, that is, the wastewater has the “five high” features. The wastewater is difficult to handle and seriously pollute the environment. (6) The corrosion of hypochlorous acid produced in the production process to the equipment is also more serious.

If the problems of waste water and waste residue cannot be effectively solved in the production of epoxide by chlorohydrination, the greater the capacity of the enterprise production device, the greater the environmental protection burden; the higher the production volume, the more serious the damage to the environment. Therefore, environmental protection has become the primary factor restricting the development of the epoxide industry. A chlorohydrination is developed and applied first in the United States to prepare epoxides. At the beginning of the application, the chlorohydrination process was widely developed and produced. In the year of 2000, the production of epoxides by chlorohydrination was prohibited in the United States due to the inability to effectively treat wastewater from the chlorohydrination process.

### SUMMARY OF THE INVENTION

First, the inventors of the present application have found through research that an unexpected technical effect is obtained by using an alkali metal hydroxide and a bipolar membrane electrodialysis technique in a method for preparing an epoxide by halohydrination.

Thus, in accordance with a first embodiment of the present invention, there is provided a method of preparing an epoxide by halohydrination, the method comprising the steps of:

(1) halohydrination: adding H₂O, a halogen(s) (preferably chlorine, bromine or iodine), and an ethylenically unsaturated compound or an olefin compound having one or more C═C double bonds to a reaction device, and performing a halohydrination reaction, so as to obtain a halohydrin;

(2) saponification: saponifying the halohydrin obtained in step (1) with an alkali metal hydroxide, and then performing a separation, so as to obtain an epoxide and an alkali metal halide;

(3) electrodialysis: the alkali metal halide obtained in the step (2) being subjected to bipolar membrane electrodialysis to obtain an alkali metal hydroxide and a hydrogen halide.

Preferably, the above method further comprises: (4) refining the epoxide (e.g., by distillation or rectification) to obtain a refined epoxide.

Preferably, the the ethylenically unsaturated compound or olefin compound having one or more C═C double bonds is selected from the group consisting of: C₂-C₆ alkenes, vinyl aromatic hydrocarbons (preferably C₈-C₂₀) and divinyl aromatic hydrocarbons (preferably C₁₀-C₂₂); More preferably, it is selected from ethylene, propylene, butylene, pentene, hexene, hexadiene and styrene.

In the present application, generally, the molar ratio of the ethylenically unsaturated compound or olefin compound to halogen (e.g., chlorine) in the step (1) is from 1:0.1 to 100, preferably from 1:0.5 to 50, more preferably 1:0.8 to 30, more preferably 1:0.9 to 20, more preferably 1:1.0 to 10, more preferably 1:1.1 to 5, more preferably 1:1.1 to 2.

Secondly, the inventors of the present application have found through research that preparation of a halogenated alcohol by using an olefin, a hydrogen halide and H₂O₂, followed by saponification, can produce an epoxide at extremely high selectivity and yield, and greatly reduce the discharge of waste water and waste residue.

Therefore, according to a second embodiment of the present invention, there is provided a method for producing a halohydrin by halohydrination, comprising the step of:

(1) halohydrination: adding a hydrogen halide, H₂O₂, and an ethylenically unsaturated compound or an olefin compound having one or more C═C double bonds to a reaction device, and performing halogenation reaction, so as to obtain a halohydrin.

In the present invention, the ethylenically unsaturated compound or olefin compound having one or more C═C double bonds is a C₂-C₃₀₀ ethylenically unsaturated compound having one or more C═C double bond, more preferably a C₂-C₂₀₀ ethylenically unsaturated compound; more preferably a C₃-C₁₅₀ ethylenically unsaturated compound; more preferably a C₃-C₁₀₀ ethylenically unsaturated compound; more preferably a C₃-C₅₀ ethylenically unsaturated compound; more preferably a C₃-C₂₀ ethylenically unsaturated Compound.

More preferably, the ethylenically unsaturated compound or olefin compound having one or more C═C double bonds is: ethylene, propylene, butylene, butadiene, pentene, pentadiene, hexene, hexadiene, heptene, heptadiene, octene, octadiene, nonene, nonadiene, decene, decadiene, undecene, dodecene, dodecadiene, dodecatriene, docosatriene, styrene, methyl styrene or divinyl benzene.

Preferably, hydrogen halide is one or more of hydrogen chloride, hydrogen bromide or hydrogen iodide.

Preferably, the molar ratio of the ethylenically unsaturated compound or the olefin compound to hydrogen halide in the step (1) is from 1:0.1 to 100, preferably from 1:0.5 to 50, more preferably from 1:0.8 to 30, still more preferably 1:0.9-20, more preferably 1:1.0-10, more preferably 1:1.1-5, still more preferably 1:1.1-2.

Preferably, the molar ratio of the ethylenically unsaturated compound or the olefin compound to H₂O₂ in the step (1) is from 1:0.1 to 100, preferably from 1:0.5 to 50, more preferably from 1:0.8 to 30, still more preferably 1:0.9-20, more preferably 1:1.0-10, more preferably 1:1.1-5, more preferably 1:1.1-2.

Preferably, the concentration (wt %) of H₂O₂ is 2-100%, preferably 5-90%, more preferably 8-80%, still more preferably 10-70%, still more preferably 15-60%, still more preferably 20-50%.

Preferably, in the present application, the catalyst is employed in the step (1). There are no restrictions on the catalyst in the present application. Generally, the catalyst is one, two or more selected from the group consisting of solid acids (such as tungstic acid, citric acid), molecular sieves (such as Ti-molecular sieves), vanadium phosphorus oxide composites, molybdenum-vanadium composite metal oxides, molybdenum-bismuth composite metals oxides, molybdenum-tungsten composite metal oxides, Salen transition metal catalysts or heteropolyacids.

In the present invention, there is no strict limit to the reaction temperature in the step (1). For example, the reaction temperature in the step (1) is from 0 to 60° C., preferably from 10 to 50° C., more preferably from 20 to 45° C. For example, the reaction temperature in the step (2) is 0-100° C., preferably 5 to 90° C., preferably 10 to 80° C., more preferably 20 to 60° C.

In the present invention, the concentration of the hydrogen halide in the step (1) may be any concentration. When a low concentration of hydrogen halide is used, the reaction rate can be well controlled; and when a high concentration of hydrogen halide is used, the reaction can proceed promptly in the direction of producing halopropanol. As long as hydrogen halide is present in the reaction system, the reaction can be carried out regardless of the concentration of the hydrogen halide, and a halohydrin can be obtained. Preferably, the concentration of the hydrogen halide is from 1 to 40%, further preferably from 2 to 30%, preferably from 3 to 20%, more preferably from 5 to 10%.

According to a third embodiment of the present invention, there is provided a method of preparing an epoxide by a halohydrination, comprising the steps of:

(1) halohydrination: adding a hydrogen halide, H₂O₂, and an ethylenically unsaturated compound or an olefin compound having one or more C═C double bonds to a reaction device, and then performing a halogenation reaction, so as to obtain a halohydrin.

(2) saponification: saponificating the halohydrin of the step (1) with an alkali metal hydroxide (preferably, sodium hydroxide, potassium hydroxide or lithium hydroxide), and then performing a separation, so as to obtain an epoxide and an alkali metal halide.

Preferably, the method further comprises: (3) electrodialysis: subjecting the alkali metal halide obtained in the step (2) to electrodialysis through a bipolar membrane to obtain an alkali metal hydroxide and hydrogen halide. The resulting hydrogen halide can be recycled to step (1).

Preferably, the method further comprises: (4) refining the epoxide to obtain a refined epoxide (such as distillation or rectification); preferably, if the epoxide obtained in the step (2) is an epoxide having a low molecular weight (e.g., an epoxide having an average molecular weight of 50 to 400, such as 70 to 300), the epoxide obtained in the step (2) is rectified to obtain a refined epoxide.

Preferably, the ethylenically unsaturated compound or olefin compound having one or more C═C double bonds is a C₂-C₃₀₀ ethylenically unsaturated compound of one or more C═C double bond, more preferably a C₂-C₂₀₀ ethylenically unsaturated compound, more preferably a C₃-C₁₅₀ ethylenically unsaturated compound, more preferably a C₃-C₁₀₀ ethylenically unsaturated compound, more preferably a C₃-C₅₀ ethylenically unsaturated compound, more preferably a C₃-C₂₀ ethylenically unsaturated compound.

More preferably, the ethylenically unsaturated compound or olefin compound having one or more C═C double bonds is: ethylene, propylene, butylene, butadiene, pentene, pentadiene, hexene, hexadiene, heptene, heptadiene, octene, octadiene, nonene, nonadiene, decene, decadiene, undecene, dodecene, dodecadiene, dodecatriene, docosatriene, styrene, methyl styrene or divinyl benzene.

Preferably, the hydrogen halide is one or more of hydrogen chloride, hydrogen bromide or hydrogen iodide.

Preferably, wherein the molar ratio of the ethylenically unsaturated compound or the olefin compound to the hydrogen halide in the step (1) is from 1:0.1 to 100, preferably from 1:0.5 to 50, more preferably from 1:0.8 to 30, still more preferably 1:0.9-20, more preferably 1:1.0-10, more preferably 1:1.1-5, still more preferably 1:1.1-2.

Preferably, wherein the molar ratio of the ethylenically unsaturated compound or the olefin compound to H₂O₂ in the step (1) is from 1:0.1 to 100, preferably from 1:0.5 to 50, more preferably from 1:0.8 to 30, still more preferably 1:0.9.20, more preferably 1:1.0-10, more preferably 1:1.1-5, more preferably 1:1.1-2.

Preferably, the concentration (wt %) of the H₂O₂ is 2-100%, preferably 5-90%, more preferably 8-80%, still more preferably 10-70%, still more preferably 15-60%, still more preferably 20-50%.

In the present invention, the concentration of the hydrogen halide in the step (1) may be any concentration. When a low concentration of hydrogen halide is used, the reaction rate can be well controlled; and when a high concentration of hydrogen halide is used, the reaction can be promptly proceeded in the direction of producing halopropanol. As long as hydrogen halide is present in the reaction system, the reaction can be carried out regardless of the concentration of the hydrogen halide, and a halohydrin can be obtained. Preferably, the concentration of the hydrogen halide is from 1 to 40%, further preferably from 2 to 30%, preferably from 3 to 20%, more preferably from 5 to 10%.

Preferably, in any one of the first embodiment, the second embodiment or the third embodiment, the catalyst is employed in the step (1). There are no restrictions on the catalyst in the present application. Generally, the catalyst is one, two or more selected from the group consisting of solid acids (such as tungstic acid, niobic acid), molecular sieves (such as Ti-molecular sieves), vanadium phosphorus oxide composites, molybdenum-vanadium composite metal oxides, molybdenum-bismuth composite metals oxides, molybdenum-tungsten composite metal oxides, Salen transition metal catalysts or heteropolyacids.

Preferably, in any one of the processes according to the third embodiment, the molar ratio of the halohydrin to the alkali metal hydroxide in the step (2) is from 1:0.1 to 50, preferably from 1:0.5 to 30, more preferably 1:0.8-20, more preferably 1:1.0-10, more preferably 1:1.1-5, still more preferably 1:1.2-4, still more preferably 1:1.5-3. Preferably, the base (i.e., alkali metal hydroxide) is used in excess relative to halohydrin.

In the present invention, there is no strict limit to the reaction temperature in the step (1) or the step (2). For example, the reaction temperature in the step (1) is from 0 to 60° C., preferably from 10 to 50° C., more preferably from 20 to 45° C. For example, the reaction temperature in the step (2) is 0-100° C., preferably 5 to 90° C., preferably 10 to 80° C., more preferably 20 to 60° C.

In any of the methods of the present invention, the reaction device in the step (1) is a single reactor, a plurality of reactors in series, a tubular reactor or a microchannel reactor.

Preferably, the reaction in step (1) is carried out in a batch, semi-continuous or continuous manner.

When the step (1) is carried out in a single reactor in a batch mode, and the catalyst is added in the step (1), the weight ratio of the ethylenically unsaturated compound or the olefin compound to the catalyst is 1:0.001-0.4, preferably 1:0.005-0.3, more preferably 1:0.01-0.2, still more preferably 1:0.015-0.1. When the step (1) is carried out in a continuous or semi-continuous manner, the catalyst can be arranged in a continuous or semi-continuous reactor(s) such as a plurality of reactors in series, a tubular reactor or a microchannel reactor, in a fixed catalyst bed manner. At this time, the amount of the catalyst is more. The weight ratio of the ethylenically unsaturated compound to the catalyst in the catalyst bed is from 1:0.001 to 0.99, preferably from 1:0.005 to 0.8, more preferably from 1:0.01 to 0.6, still more preferably from 1:0.015 to 0.4.

Preferably, the catalyst of the present invention may be one or more of a molecular sieve containing Ti heteroatom, an HTS molecular sieve, and a molecular sieve containing a Zr heteroatom and having a MFI structure.

TS-1 is a titanium-silicon molecular sieve, belonging to the Pentasil-type heteroatomic molecular sieve, orthorhombic system, and its synthesis method is referred to CN201410812216.8. TS-2 molecular sieve has MEL topology and has good catalytic oxidation performance, and its synthesis method is referred to CN200910013070.X. Ti-MWW exhibits better catalytic oxidation performance in the epoxidation of linear olefins and cyclic olefins and high trans-selectivity in the asymmetric epoxidation of olefins, and its synthesis method is referred to CN200710037012.1. Ti-Beta molecular sieve has a three-dimensional staggered 12-membered ring channel structure and stronger acidity, and has good catalytic performance in heterogeneous catalytic reaction. The specific synthesis method is based on “Synthesis of Beta Molecular Sieve and Research of its Catalytic Performance”, author: Liu Shuping, East China Normal University.

Ti-SBA-15 is a kind of mesoporous molecular sieve, Ti-SBA-15 has a two-dimensional hexagonal through-hole structure, and its synthesis method is referred to CN201110211854.0.

HTS molecular sieves include HTS-1 molecular sieves, HTS-2 molecular sieves, and HTS-3 molecular sieves. The acidic sites on the surface of HTS-1 molecular sieve have higher catalytic activity, so their diffusion performance and the solvent polarity greatly affect the reactivity. The specific synthetic method of HTS-1 molecular sieve is referred to “Probe Reactions Catalyzed by Surface Acid Sites of HTS-1”, author: Liu Xuanyan, Yin Dulin, Zhu Huayuan, Shen Gang, Chinese Journal of Catalysis. The titanium-silica molecular sieve HTS-2 has the same catalytic oxidation activity, selectivity, and general physicochemical properties as the titanium-silica molecular sieve HTS-1. The specific synthetic method of HTS-2 molecular sieve is referred to “Synthesis of titanium-silica molecular sieve HTS-2”, author: Zhu Bin, Lin Min, Shu Xingtian, Wang Yuqing. The single-crystal multi-hollow titanium-silica molecular sieve catalyst HTS-3, due to uniform distribution of titanium on the surface of the molecular sieve, can shorten crystallization time of the molecular sieve and improve synthesis stability and repeatability. HTS-3 molecular sieve catalyst has more active centers, better catalysis and is more conducive to the diffusion of reactant molecules. Its specific synthesis is referred to “Development of single crystal multi-hollow HTS-3 molecular sieve catalyst”, Petroleum Refining and Chemical Industry.

Molecular sieves containing Zr heteroatoms and having MFI structure are typical solid Lewis acid catalysts, which exhibit good catalytic activity in the reaction. The molecular sieve containing Zr heteroatom and having MFI structure is a kind of metal element zirconium incorporated into the MFI structure (orthogonal system, mesh straight hole) molecules, and ZSM-5, Silicalite-1 and the like are common. ZSM-5 molecular sieves were prepared by the method of Chinese Patent Application No. 200510029462.7. Silicalite-1 molecular sieves were prepared by the method of Chinese Patent Application No. 201010220796.3.

Preferably, the halohydrination reaction is carried out in a bubble column or turbulent tube reactor without internal parts, and the reaction pressure may be higher than normal pressure, or normal pressure, or near normal pressure. For example, at a temperature of 0 to 60° C., the conversion of an olefin (for example, propylene) reaches 97%, and hydrogen peroxide is substantially completely converted.

The saponification reaction is carried out in a steel column reactor, and the upper part is designed as a sieve tray column. Steam enters the bottom of the column and the resulting crude propylene oxide is blown out from the top of the column. The saponification temperature is controlled at 0 to 100° C., and the pressure at the top of the column is normal pressure or negative pressure. The key to the design and operation of the saponification reactor is that the aqueous solution of the halohydrin before entering the saponification column must be thoroughly mixed with lye (alkali solution) and its pH is controlled so that the propylene oxide formed flows out from the top of the column as quickly as possible.

Take a method of producing propylene oxide from propylene as an example: Since the conventional chlorohydrination uses lime for saponification, all of the chlorine is eventually consumed in the form of CaCl₂, producing a large amount of sewage containing CaCl₂ and organic chloride. It is shown from calculation that for every 1 ton of propylene oxide produced, about 2.1 tons of CaCl₂ slag and at least 43 tons of wastewater are produced.

(1) A reaction system combining a tubular reactor and a column reactor is used to reduce the load on the chlorohydrination column.

(2) The saponification column is operated under reduced pressure, and the amount of steam used is small.

(3) In the rectification step, a front distillation column and a rectification column are provided, and the two columns respectively produce propylene oxide for preparing propylene glycol and propylene oxide for preparing polyether.

(4) Dichloroisopropyl ether in the waste water produced by the saponification process is separated by a dispersing column having special trays to reduce the biological oxygen content and the chemical oxygen content in the waste liquid.

In the prior art, the process of preparing an epoxide by halohydrination (taking chlorine as an example):

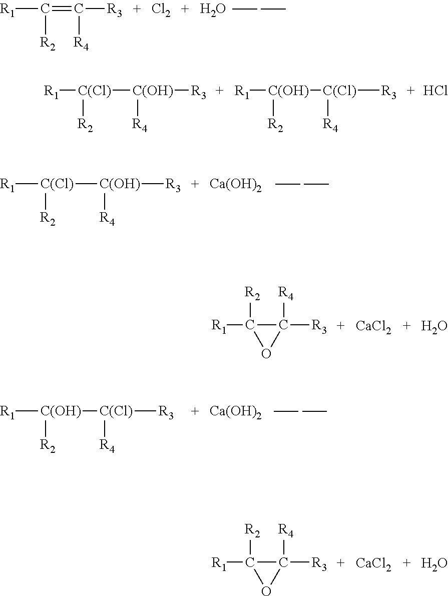

The invention provides a process for preparing an epoxide by novel halohydrination (taking hydrogen chloride as an example):

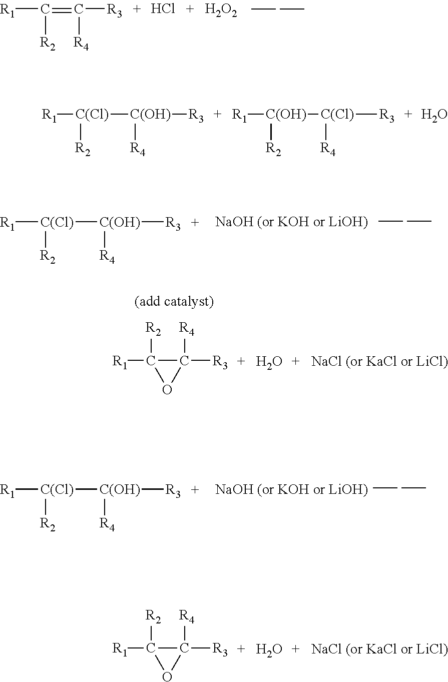

Wherein, R₁, R₂, R₃, and R₄ each may be a saturated or unsaturated structure containing a C₂-C₃₀₀ linear chain, branched chain or ring (heterocyclic or benzene ring) and the like, which may have other atoms such as a halogen, an oxygen atom, a sulfur atom, a nitrogen atom or the like For example, each of R₁, R₂, R₃, and R₄ may be one of H, CH₃—, CH₃CH₂—, CH₃CH₃CH₃CH₂—, CH₃CH₂═CH₂—, CH₂(Cl)CH₂—,

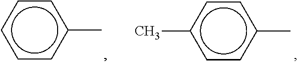

and the like. For example, R₁, R₂, R₃, and R₄ are all H; or R₁ is CH₃—, R₂ is H, R₃ is H, and R₄ is H; or R₁ is CH₃CH₂—, R₂ is CH₃—, R₃ is H, and R₄ is H; or R₁ is CH₂(Cl)CH₂—, R₂ is CH₃—, R₃ is H, and R₄ is CH₃—; or R₁ is

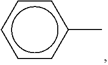

R₂ is H, R₃ is H, and R₄ is CH₃—.

NaCl (KaCl or LiCl) was electrodialyzed by a bipolar membrane to become NaOH (KOH or LiOH) and HCl. The alkali metal hydroxide and hydrogen halide produced by electrodialysis using bipolar membrane can be circulated.

Bipolar membrane is a new type of ion exchange composite membrane, which is usually composed of a cation exchange layer (N type membrane), an interface hydrophilic layer (catalytic layer) and an anion exchange layer (P type membrane). It is really a reaction film. Under the action of a direct current electric field, the bipolar membrane dissociates water and respectively obtains hydrogen ions and hydroxide ions on two sides of the membrane. By utilizing this feature, a bipolar membrane electrodialysis system combining a bipolar membrane with other anion-cation exchange membranes can convert a salt in an aqueous solution into a corresponding acid and base without introducing a new component. The method is called bipolar membrane electrodialysis.

The bipolar membrane electrodialysis apparatus used in the present application is not particularly limited. However, it is preferred to employ a bipolar membrane electrodialysis apparatus comprising: 1) a membrane stack composed of a bipolar membrane, an anion membrane, a cation membrane, a separator, and a polar plate as core components, and 2) auxiliary devices including water tanks, flow meters, pumps, pipes, and the like. The auxiliary devices also include a rectifier cabinet. A stainless steel frame is used in the core components. The number of membranes and separators can vary depending on the particular amount of treatment (the volume of the salt solution).

Bipolar membrane, one of the electro-driven membranes, mainly provides H⁺ ions and OH⁻ ions under electric field force. One side of the membrane is an anion surface, the other side is a cation surface, and the middle layer between the anion surface and the cation surface is an aqueous layer. Under the action of the applied DC electric field force, the H₂O in the aqueous layer splits into H⁺ ions and OH⁻ ions, and H⁺ ions and OH⁻ ions migrates respectively through the anion surface and the cation surface to the corresponding solutions on both sides, so the role of the bipolar membrane is provided H⁺ ion and OH⁻ ion source by the electric field force.

In the present invention, when the salty wastewater is treated by bipolar membrane electrodialysis, the corresponding acid and base are obtained.

### Advantages of the Invention

In the prior art, the epoxide is produced by the chlorohydrin method, and a large amount of calcium chloride-containing wastewater is produced. The wastewater also contains raw materials that are not completely reacted during the reaction or intermediate organic products, and also contains unseparated epoxide. These organic wastes are difficult to degrade and are very difficult to handle, and no effective treatment has been found. The main reason for the ban on the chlorohydrination process in the United States in 2000 is that, calcium chloride wastewater which contains organic waste that is difficult to degrade, is difficult to handle. Some people use the electrolytic method to treat the wastewater generated by the chlorohydrination process. Because the waste water contains organic waste, it is very difficult to handle, and the organic waste in the waste water is very sensitive to the ion-exchange membrane electrolysis system, and the electrolytic equipment is damaged. At the same time, the electrolysis of waste water will consume a lot of energy and the benefits therefrom are minimal. The biggest problem is that the electrolysis of wastewater produces chlorine and hydrogen, and in the presence of organic matter, it is very dangerous and easy to explode.

In the method according to the first embodiment of the present invention, the bipolar membrane electrodialysis technique used in the present invention is one for treating a salt which is subjected to bipolar membrane electrodialysis to produce an acid and a base. Bipolar membrane electrodialysis has previously been used to treat wastewater containing sodium chloride, but bipolar membrane electrodialysis has never been applied to epoxide production processes. This is due to inherent prejudice, as the wastewater prepared by the chlorohydrination contains organic waste which is difficult to degrade, ordinary organic waste is sensitive to the ion membrane and the treatment effect is very poor. After numerous experiments and experiments, the inventors found that the bipolar membrane electrodialysis treatment of the epoxide wastewater by the chlorohydrination is excellent. The wastewater containing organic waste produced from the chlorohydrination does not affect the process of bipolar membrane electrodialysis and the process can handle the salt in the wastewater very well. The invention skillfully utilizes the features of the organic matter in the wastewater resulted from the halohydrination for preparing epoxide, can well apply the bipolar membrane electrodialysis technology to the preparation of the epoxide, and realize the clean production of epoxide and solve the problem of the wastewater resulted from the halohydrination for preparing epoxide being difficult to handle. As the problem of wastewater treatment is overcome, so the advantages of the preparation of epoxide by the halohydrination are quite obvious. The input for halohydrination is small, the conversion rate is high, and it is easy to control. Therefore, the invention introduces a bipolar membrane electrodialysis technology to subvert the traditional halohydrination method for preparing an epoxide, completely solves the problem of waste water containing organic matters, and enables halohydrination to achieve clean production for epoxide while efficiently preparing epoxide.

In the second and third embodiments of the present invention, the acid and the base are the raw materials of the present embodiment, and the acid and the alkali produced by the bipolar membrane electrodialysis can be used as a raw material again, thereby form a cycle to achieving production in a cleaning, recycling and no-waste manner. It turns the original waste from the former stage into the current raw materials in the later stage, which can save materials, reduce waste emissions, and protect the environment.

Compared with the electrolysis process, the energy consumed by the electrodialysis of the bipolar membrane is greatly reduced, and the effect is improved. At the same time, the acid and alkali produced by bipolar membrane electrodialysis are not dangerous, and overcome the problem of the danger of electrolysis process and easy explosion. The most important thing is that the electrolytic process treats wastewater but the organic impurities in the wastewater can not be treated and can only be discharged. Such organic waste is difficult to degrade and greatly pollute the environment. If the bipolar membrane electrodialysis is used to treat such wastewater, the organic impurities in the wastewater can be reduced to raw materials or directly to obtain products, and thus the salt in the wastewater is treated while the organic waste in the waste water is also treated, so as to turn the organic waste into raw materials or products, to make full use of the materials, and at the same time to achieve waste zero emission and a good protection of the environment.

In the second and third embodiments of the present invention, a hydrogen halide (e.g., hydrogen chloride) is used in place of the chlorine in the prior art. Since the chlorine gas is in a gaseous state, a part of the olefin compound is also in a gaseous state, and side addition reaction easily occurs in a gas phase environment. The present invention achieves a good solution to the problem of side addition reaction by replacing halogen with hydrogen halide. At the same time, due to a large amount of heat is released from the side addition reaction, this makes the reaction more risk and easily to explode, but this problem is avoided in the present invention, and also the process is safe and such raw materials are highly utilized. At the same time, by using hydrogen halide instead of chlorine, the transportation and storage of hydrogen halide is easier and safer, and more conducive to production of epoxide.

Compared with the prior art, the technical solution of the present invention has the following beneficial technical effects:

1. Overcoming the problems of by-products (chlorine gas, calcium chloride, side reaction products) in the prior art: (1) not using Cl₂ as a raw material, avoiding the explosion hazard caused by accumulation of trace oxygen in chlorine gas with the continuous consumption of Cl₂; (2) no Cl₂ participating in the reaction, which greatly reduces the intermediate side reaction and reduces the production of organic by-products; (3) in the saponification process, the amount of alkali is greatly reduced, which is only about ½ of the existing process.

2. In the prior art, in order to reduce the occurrence of side reactions, the concentration (mass fraction) of the halohydrin prepared is generally controlled to be less than 4.5%; whereas in the novel halohydrination process of the present invention, the concentration of the halohydrin is not limited due to the little side reaction. 1) Saving energy consumption in the saponification process, the saponification energy consumption of the invention is only about 1/10 of the existing process; 2) saving water resources, the water consumption of the invention is less than 1/10 of the original process; at the same time, the discharge of wastewater is greatly reduced and the wastewater is easy to handle.

3. The bipolar membrane electrodialysis technology is used to treat organic wastewater, and the produced NaOH (or KOH or LiOH) and HCl can be reused.

4. Various olefins are widely adaptable, and a long-chain olefinic compound can be used as long as it is an olefin having an olefinic bond.

### BRIEF DESCRIPTION OF THE DRAWINGS

FIG. 1 is a schematic diagram of a bipolar membrane electrodialysis process of the present invention.

FIG. 2 is a schematic view showing the operation of the bipolar membrane of the present invention.

Reference numerals: 1, 2, 3: mixing containers.

### EMBODIMENTS FOR CARRYING OUT THE INVENTION

The invention is further illustrated by the following examples, which do not limit the scope of the invention.

The product was qualitatively analyzed by Agilent 7890/5975C-GC/MSD gas chromatography mass spectrometer. The product was quantitatively analyzed by Agilent 6890N gas chromatograph and external standard method.

Based on the results of the analysis, the following objective function is defined as an indicator.

Olefin conversion rate:

$${C}_{olefin} = \frac{{n}_{0 - olefin} - {n}_{olefin}}{{n}_{0 - olefin}} \times 100 \%$$

Halohydrin selectivity:

$${S}_{halohydrin} = \frac{{n}_{halohydrin}}{{n}_{0 - olefin} - {n}_{olefin}} \times 100 \%$$

Halohydrin yield: Y_{halohydrin}=C_{olefin}×S_{olefin}

Epoxide yield:

$${Y}_{epoxide} = \frac{{n}_{epoxide}}{{n}_{0 - olefin}} \times 100 \%$$

In the formula, C is a conversion ratio, S is a selectivity, Y is a yield, n is the amount of the substance after the reaction, and n0 is the amount of the starting material.

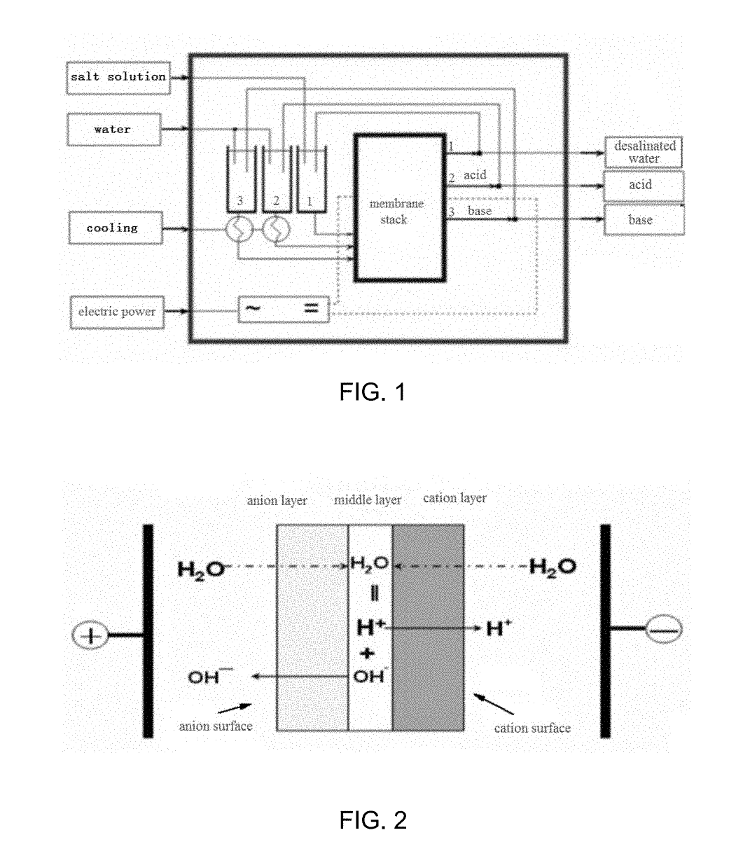

The bipolar membrane electrodialysis device is shown in FIGS. 1 and 2.

A bipolar membrane electrodialysis apparatus comprises: 1) a membrane stack composed of a bipolar membrane, an anion membrane, a cation membrane, a separator, and a plate as core components, and 2) auxiliary devices including a water tank, flow meters, pumps, pipes, etc. The auxiliary devices further include a rectifier cabinet. A stainless steel frame is used in the core components. The number of membranes and separators can vary depending on the particular amount of treatment (the volume of the salt solution).

Bipolar membrane, one of the electro-driven membranes, mainly provides H+ ions and OH-ions under electric field force. One side of the membrane is an anion surface, the other side is a cation surface, and the middle layer between the anion surface and the cation surface is an aqueous layer. Under the action of the applied DC electric field force, the H₂O in the aqueous layer splits into H+ ions and OH− ions, and H+ ions and OH− ions migrates respectively through the anion surface and the cation surface to the corresponding solutions on both sides, so the role of the bipolar membrane is to provide H+ ion and OH− ion source by the electric field force.

The performances of the bipolar membrane are as follows:

| | | exchange capacity | | | |
|---|---|---|---|---|---|
| | Membrane | (milli-equivalents per | water | Trans-membrane | bursting |
| | thickness | gram of dry | content | Voltage* | strength |
| Appearance | (mm) | membrane) | (%) | (V) | (MPa) |
| brownish black on | 0.16-0.23 | 1.4-1.8 through the | 35-40 | 0.6-1.6 | >0.25 |
| the cation surface; | | cation surface | | | |
| light grey on the | | 0.7-1.1 through the | | | |
| anion surface | | anion surface | | | |

*Measured in a 0.5 Mol Na₂SO₄ solution at 25° C., under current density of 10-100 mA/cm².

### Example A

1) Halohydrination: water, chlorine (Cl₂) and propylene are added to a tubular reactor in which a fixed tungstic acid catalyst bed is packed in the tube, and chlorohydrination reaction is carried out at a temperature of 45° C. The residence time in the tubular reactor was 30 minutes, wherein the flow rates of chlorine (Cl₂) and propylene respectively are such that the molar ratio of liquid chlorine (Cl₂) to propylene was 3.4:1, and the amount of water introduced was 99 times the mass of propylene. Halohydrin (a mixture of 2-chloropropan-1-ol and 1-chloropropan-2-ol) is obtained, which contains 9 wt % of a halogenated hydrocarbon by-product.

2) Saponification: The halohydrin obtained in the step 1) is subjected to a saponification reaction with sodium hydroxide, and the resulting mixture is performed a separation to obtain a propylene oxide organic phase and a sodium chloride solution. The saponification reaction is carried out in a steel column reactor, and the upper part is designed as a sieve tray column. Steam enters the bottom of the column and the resulting crude propylene oxide is blown out from the top of the column. The saponification temperature is controlled at 90 to 105° C.

3) Electrodialysis: The sodium chloride solution obtained in the step 2) was subjected to bipolar membrane electrodialysis to obtain sodium hydroxide and HCl.

4) Refining of propylene oxide:

The crude propylene oxide obtained in the step 2 was subjected to rectification to obtain propylene oxide of high purity (99.0 wt %).

The presence of halogenated hydrocarbon by-products has little or no effect on the effect of bipolar membrane electrodialysis.

After analysis, it is found that bipolar membrane electrodialysis produces acid and base. In the bipolar membrane electrodialysis process, when the olefin compound encounters acid, the olefin mixture reacts with the acid to form a halohydrin, which is an intermediate product of the present invention. Therefore, it can be directly utilized. When the olefin compound encounters a base, they do not react and do not affect bipolar membrane electrodialysis. When the halohydrin encounters the acid, it does not change and does not affect the bipolar membrane electrodialysis. When the halohydrin encounters the base, the reaction takes place to form an epoxide, which is exactly the product of the present invention (the desired substance). The product can be obtained by separation. When the epoxide encounters an acid, a reaction and ring opening takes place, and a halohydrin product is formed. The halohydrin is an intermediate product of the present invention, so that it can be directly used. When the epoxide encounters a base, it does not react and does not affect the bipolar membrane electrodialysis.

### Example 1

1) Halohydrination: In a tubular reactor (wherein a fixed tungstic acid catalyst bed was packed in the tube), 70 wt % hydrogen peroxide H₂O₂, 35 wt % HCl solution (hydrochloric acid) and propylene were added, and chlorohydrination reaction was carried out at a temperature of 45° C., wherein the flow rates of 70 wt % hydrogen peroxide (H₂O₂), 35 wt % HCl solution and propylene respectively were such that the molar ratio of H₂O₂ to HCl to propylene was about 1.2:1.2:1. Halohydrin (a mixture of 2-chloropropan-1-ol and 1-chloropropan-2-ol) was obtained.

### Comparative Example 1

1) Halohydrination: In a tubular reactor (wherein a fixed tungstic acid catalyst bed was packed in the tube), water, chlorine and propylene are added, and the chlorohydrination was carried out at a temperature of 45° C., wherein the flow rates of water, chlorine gas and propylene respectively should be such that the molar ratio of H₂O to chlorine to propylene was about 88:3.6:1. Halohydrin (a mixture of 2-chloropropan-1-ol and 1-chloropropan-2-ol) was obtained.

### Example 2

1) Halohydrination: Tungstic acid catalyst, a 35 wt % hydrogen peroxide (H₂O₂), a 20 wt % HCl solution (hydrochloric acid) and propylene were added to a column reactor to carry out a chlorohydrination reaction at a temperature of 35° C., wherein the mass ratio of the tungstic acid catalyst to propylene was 0.05:1, and the added amounts of 35 wt % hydrogen peroxide (H₂O₂), 20 wt % HCl solution and propylene respectively should be such that the molar ratio of H₂O₂ to HCl to propylene was about 1.5:1.1:1. Halohydrin (a mixture of 2-chloropropan-1-ol and 1-chloropropan-2-ol) was obtained.

### Comparative Example 2

1) Halohydrination: Tungstic acid catalyst, water, chlorine and propylene were added to a column reactor, and the chlorohydrination reaction was carried out at a temperature of 35° C., wherein the mass ratio of the tungstic acid catalyst to propylene was 0.05:1, and the added amounts of water, chlorine gas and propylene respectively should be such that the molar ratio of water to chlorine to propylene is about 80:3.3:1. Halohydrin (a mixture of 2-chloropropan-1-ol and 1-chloropropan-2-ol) was obtained.

### Comparative Example 3

Halohydrination: 65 wt % hydrogen peroxide (H₂O₂), a 35 wt % HCl solution (hydrochloric acid) and propylene were added in a tubular reactor, and a chlorohydrination reaction was carried out at a temperature of 45° C., wherein the flow rates of the 65 wt % hydrogen peroxide (H₂O₂), 35 wt % HCl solution and propylene respectively should be such that the molar ratio of H₂O₂ to HCl to propylene is about 1.2:1.2:1. Halohydrin (a mixture of 2-chloropropan-1-ol and 1-chloropropan-2-ol) was obtained.

### Comparative Example 4

Halohydrination: To a tubular reactor (in which a fixed tungstic acid catalyst bed was packed in a tube) were added a 35 wt % HCl solution (hydrochloric acid) and propylene, and a chlorohydrination reaction was carried out at a temperature of 45° C., wherein the flow rates of the 35 wt % HCl solution and propylene respectively should be such that the molar ratio of HCl to propylene was about 1.2:1.

### Example 3

1) Halohydrination: In a tubular reactor (wherein a fixed tungstic acid catalyst bed was packed in the tube), 70 wt % hydrogen peroxide (H₂O₂), 35 wt % HCl solution (hydrochloric acid) and propylene were added, and chlorohydrination reaction was carried out at a temperature of 45° C., in which the flow rates of 70 wt % hydrogen peroxide (H₂O₂), 35 wt % HCl solution and propylene respectively should be such that the molar ratio of H₂O₂ to HCl to propylene was about 1.2:1.2:1. Halohydrin (a mixture of 2-chloropropan-1-ol and 1-chloropropan-2-ol) was obtained.

2) Saponification: The halohydrin obtained in the step 1) was subjected to a saponification reaction with sodium hydroxide to obtain a propylene oxide organic phase and a sodium chloride solution. The saponification reaction was carried out in a steel column reactor, and the upper part was designed as a sieve tray column. Steam entered the bottom of the column and the resulting crude propylene oxide was blown out from the top of the column. The saponification temperature was controlled at 60 to 70° C.

3) Electrodialysis: The sodium chloride solution obtained in step 2) was subjected to bipolar membrane electrodialysis (TRPB8040-I type, manufactured and sold by Beijing Tingrun Membrane Technology Development Co., Ltd., the applied transmembrane voltage was 1.3V, and the working temperature was 20-30° C.), sodium hydroxide and HCl were obtained.

4) Refining of propylene oxide:

The crude propylene oxide product obtained in the step 2 was subjected to rectification to obtain propylene oxide of high purity (99.9 wt %).

### Example 4

1) Halohydrination: Tungstic acid catalyst, a 35 wt % hydrogen peroxide (H₂O₂), a 20 wt % HCl solution (hydrochloric acid) and propylene were added to a column reactor, and chlorohydrination reaction was carried out at a temperature of 35° C., wherein the mass ratio of the tungstic acid catalyst to propylene was 0.05:1, and the added amounts of 35 wt % hydrogen peroxide (H₂O₂), 20 wt % HCl solution and propylene respectively should be such that the molar ratio of H₂O₂ to HCl to propylene was about 1.5:1.1:1. Halohydrin (a mixture of 2-chloropropan-1-ol and 1-chloropropan-2-ol) was obtained.

2) Saponification: The halohydrin obtained in the step 1) was subjected to a saponification reaction with sodium hydroxide, so as to obtain a propylene oxide organic phase and a sodium chloride solution. The saponification reaction was carried out in a steel column reactor, and the upper part was designed as a sieve tray column. Steam entered the bottom of the column and the resulting crude propylene oxide was blown out from the top of the column. The saponification temperature was controlled at 30 to 40° C.

3) Electrodialysis: The sodium chloride solution obtained in the step 2) was subjected to bipolar membrane electrodialysis to obtain sodium hydroxide and HCl.

4) Refining of propylene oxide:

The crude propylene oxide product obtained in the step 2 was subjected to rectification to obtain propylene oxide of high purity (99.9 wt %).

### Example 5

Example 3 was repeated except that the HBr solution was used instead of the HCl solution.

### Example 6

Example 3 was repeated except that the tube of the tubular reactor was packed with a fixed bed of niobic acid catalyst, i.e., niobic acid was used instead of tungstic acid.

### Example 7

Example 3 was repeated except that the molybdenum-bismuth composite metal oxide Mo₁₂Bi_{1.6}Fe_{2.2}Co_{5.5}Ni_{2.5}Sb_{0.5}Zn_{0.3}K_{0.1}O_{32.8} was used.

### Example 8

Example 3 was repeated except that the tube of the tubular reactor was packed with a fixed catalyst bed of TS-1, i.e., TS-1 was used instead of tungstic acid. For the synthesis method of TS-1, please refer to Example 1 of Chinse Patent Appilcation CN201410812216.8.

### Example 9

Example 3 was repeated except that the catalyst bed of TS-2 was used. For the synthesis method of TS-2, please refer to the Embodiment of CN200910013070. X (only one Example therein).

### Example 10

Example 3 was repeated except that a fixed Ti-MWW catalyst bed was packed in the tube of the tubular reactor. For the synthesis method of Ti-MWW, please refer to Example 1 of CN200710037012.1.

### Example 11

Example 3 was repeated except that a catalyst bed of Ti-Beta was used.

### Example 12

Example 3 was repeated except that a catalyst bed of Ti-SBA-15 was used. For the synthesis method of Ti-SBA-15, please refer to Example 1 of CN201110211854.0.

### Example 13

Example 3 was repeated except that the catalyst bed of HTS-1 was used.

### Example 14

Example 3 was repeated except that the catalyst bed of HTS-2 was used.

### Example 15

Example 3 was repeated except that the catalyst bed of HTS-3 was used.

### Example 16

Example 3 was repeated except that the catalyst bed of ZSM-5 molecular sieve was used. The ZSM-5 molecular sieve was prepared by the method of Examples 1-3 of CN 200510029462.7.

### Example 17

Example 3 was repeated except that the catalyst bed of Silicalite-1 molecular sieve was used. The Silicalite-1 molecular sieve was prepared by the method of Example 1 of CN201010220796.3.

### Example 18

Example 3 was repeated except that ethylene was used instead of propylene.

### Example 19

Example 3 was repeated except that divinylbenzene was used in place of propylene.

### Example 20

Example 3 was repeated except that octene-1 was used instead of propylene.

### Example 21

Example 3 was repeated except that styrene was used instead of propylene.

### Example 22

Example 3 was repeated except that the sodium hydroxide in step (2) was replaced with potassium hydroxide.

### Example 23

Example 3 was repeated except that the flow rates of 65 wt % hydrogen peroxide (H₂O₂), 35 wt % HCl solution and propylene respectively in step (1) was such that the molar ratio of H₂O₂ to HCl to propylene was about 0.8:0.3:1.

### Example 24

Example 3 was repeated except that the flow rates of 20 wt % hydrogen peroxide (H₂O₂), 35 wt % HCl solution and propylene respectively in step (1) was such that the molar ratio of H₂O₂ to HCl to propylene was about 0.9:1.1:1.

### Example 25

Example 3 was repeated except that the reaction temperature in the step (1) was 20° C.

### Example 26

Example 3 was repeated except that the temperature in step (2) was controlled at 50° C.

Table 1 Reaction conditions and reaction results of Examples 1-26 and Comparative Examples 1-4

| | | H₂O₂ | Catalyst | | | | | | |
|---|---|---|---|---|---|---|---|---|---|
| | | (or | amount | | | | | | |
| | | Water): | (based | | | | | | |
| | | hydrogen | on | | | | | | |
| | | halide | the | | | | | | |
| | | (or | mass | | | | | | |
| | | halogen): | of | halohydrination | Reaction | Olefin | Halohydrin | Halohydrin | Epoxide |
| | | olefin | olefin), | temperature, | time, | conversion, | selectivity, | yield, | yield, |
| Items | Catalyst | (mole) | % | ° C. | min | % | % | % | % |
| Ex.1 | tungstic acid | 1.2:1.2:1 | bed | 45 | 40 | 100.00 | 96.34 | 96.34 | / |
| Comp. | tungstic acid | 88:3.6:1 | bed | 45 | 40 | 78.84 | 78.17 | 61.63 | / |
| Ex.1 | | | | | | | | | |
| Ex.2 | tungstic acid | 1.5:1.1:1 | 5 | 35 | 10 | 100.00 | 96.01 | 96.01 | / |
| Comp. | tungstic acid | 80:3.3: | 5 | 35 | 10 | 75.13 | 74.86 | 56.24 | / |
| Ex.2 | | | | | | | | | |
| Comp. | None | 1.2:1.2:1 | 0 | 45 | 40 | 1.02 | 31.24 | 0.32 | / |
| Ex.3 | | | | | | | | | |
| Comp. | tungstic acid | 0:1.2:1 | bed | 45 | 40 | 0 | 0 | 0 | / |
| Ex.4 | | | | | | | | | |
| Ex.3 | tungstic acid | 1.2:1.2:1 | bed | 45 | 40 | 100.00 | 96.34 | 96.34 | 93.73 |
| Ex.4 | tungstic acid | 1.5:1.1:1 | 5 | 35 | 10 | 100.00 | 96.01 | 96.01 | 93.11 |
| Ex.5 | tungstic acid | 1.2:1.2:1 | bed | 45 | 40 | 100.00 | 96.33 | 96.33 | 93.72 |
| Ex.6 | niobic acid | 1.2:1.2:1 | bed | 45 | 40 | 100.00 | 96.41 | 96.41 | 93.89 |
| Ex.7 | Mo₁₂Bi₁₆Fe_{2.2} | 1.2:1.2:1 | bed | 45 | 40 | 100.00 | 96.44 | 96.44 | 93.90 |
| | Co_{5.5}Ni_{2.5}Sb_{0.5} | | | | | | | | |
| | Zn_{0.3}K_{0.1}O_{32.8} | | | | | | | | |
| Ex .8 | TS-1 | 1.2:1.2:1 | bed | 45 | 40 | 100.00 | 98.16 | 98.16 | 92.34 |
| Ex.9 | TS-2 | 1.2:1.2:1 | bed | 45 | 40 | 100.00 | 98.19 | 98.19 | 96.31 |
| Ex.10 | Ti-MWW | 1.2:1.2:1 | bed | 45 | 40 | 100.00 | 98.21 | 98.21 | 96.58 |
| Ex.11 | Ti-Beta | 1.2:1.2:1 | bed | 45 | 40 | 100.00 | 98.22 | 98.22 | 97.00 |
| Ex.12 | Ti-SBA-15 | 1.2:1.2:1 | bed | 45 | 40 | 100.00 | 98.25 | 98.25 | 97.22 |
| Ex.13 | HTS-1 | 1.2:1.2:1 | bed | 45 | 40 | 100.00 | 98.52 | 98.52 | 95.89 |
| Ex.14 | HTS-2 | 1.2:1.2:1 | bed | 45 | 40 | 100.00 | 98.89 | 98.89 | 96.99 |
| Ex.15 | HTS-3 | 1.2:1.2:1 | bed | 45 | 40 | 100.00 | 99.01 | 99.01 | 97.15 |
| Ex.16 | ZSM-5 | 1.2:1.2:1 | bed | 45 | 40 | 100.00 | 98.83 | 98.83 | 94.41 |
| Ex.17 | Silicalite-1 | 1.2:1.2:1 | bed | 45 | 40 | 100.00 | 98.83 | 98.83 | 96.01 |
| Ex.18 | tungstic acid | 1.2:1.2:1 | bed | 45 | 40 | 100.00 | 95.18 | 95.18 | 92.77 |
| | | (ethylene) | | | | | | | |
| Ex.19 | tungstic acid | 1.2:1.2:1 | bed | 45 | 40 | 100.00 | 96.30 | 96.30 | 92.28 |
| | | (diyinyl | | | | | | | |
| | | benzene) | | | | | | | |
| Ex.20 | tungstic acid | 1.2:1.2:1 | bed | 45 | 40 | 100.00 | 96.46 | 96.46 | 92.37 |
| | | (octene-1) | | | | | | | |
| Ex.21 | tungstic acid | 1.2:1.2:1 | bed | 45 | 40 | 100.00 | 96.39 | 96.39 | 92.42 |
| | | (styrene) | | | | | | | |
| Ex.22 | tungstic acid | 1.2:1.2:1 | bed | 45 | 40 | 100.00 | 96.34 | 96.34 | 92.28 |
| Ex.23 | tungstic acid | 0.8:0.3:1 | bed | 45 | 40 | 30.00 | 96.11 | 28.83 | 91.22 |
| Ex.24 | tungstic acid | 0.9:1.1:1 | bed | 45 | 40 | 90.00 | 96.19 | 86.57 | 91.89 |
| Ex.25 | tungstic acid | 1.2:1.2:1 | bed | 20 | 2 | 100.00 | 96.30 | 96.30 | 92.19 |
| Ex.26 | tungstic acid | 1.2:1.2:1 | bed | 45 | 40 | 100.00 | 96.34 | 96.34 | 92.18 |

By catalyst amount being “a bed”, it is meant that the catalyst is disposed in the reactor in the form of a fixed catalyst bed, such as a plurality of reactors in series, tubular reactors or microchannel reactors. The weight ratio of ethylenically unsaturated compound to catalyst in the catalyst bed was 1:0.3.

1-15. (canceled)

16. A method for preparing a halohydrin, said method comprising the step of:
(1) halohydrination: adding a hydrogen halide, H₂O₂, and an ethylenically unsaturated compound or an olefin compound having one or more C═C double bonds to a reaction device, and performing halohydrination reaction, so as to obtain a halohydrin, wherein molar ratio of the ethylenically unsaturated compound or the olefin compound to the hydrogen halide is from 1:0.9-20,
wherein in said step (1), a catalyst is used, which is one, two or more selected from the group consisting of solid acids, molecular sieves, vanadium phosphorus oxide composites, molybdenum-vanadium composite metal oxides, molybdenum-bismuth composite metal oxides, molybdenum-tungsten composite metal oxides, Salen transition metal catalysts or heteropolyacids.

17. The method according to claim 16, wherein said method further comprising
(2) saponification: saponificating of the halohydrin obtained in the step (1) with an alkali metal hydroxide (preferably, sodium hydroxide, potassium hydroxide or lithium hydroxide) and then performing a separation, so as to obtain an epoxide and an alkali metal halide, wherein molar ratio of the halohydrin to the alkali metal hydroxide is from 1:0.8-20,
wherein in step (1), a catalyst is used, which is one, two or more selected from the group consisting of solid acids, molecular sieves, vanadium phosphorus oxide composites, molybdenum-vanadium composite metal oxides, molybdenum-bismuth composite metal oxides, molybdenum-tungsten composite metal oxides, Salen transition metal catalysts or heteropolyacids.

18. The method according to claim 17, wherein said method further comprising:
(3) electrodialysis: subjecting the alkali metal halide obtained in the step (2) to electrodialysis through a bipolar membrane to obtain an alkali metal hydroxide and hydrogen halide.

19. The method according to claim 18, wherein the method further comprising:
(4) refining the epoxide to obtain a refined epoxide.

20. The method according to claim 16, wherein the catalyst is one, two or more selected from the group consisting of tungstic acid, niobic acid and Ti-molecular sieves.

21. The method according to claim 16, wherein the ethylenically unsaturated compound or olefin compound having one or more C═C double bonds is a C₂-C₅₀ ethylenically unsaturated compound having one or more C═C double bonds; and/or
the hydrogen halide is one or more of hydrogen chloride, hydrogen bromide or hydrogen iodide, and the concentration of the hydrogen halide is from 5 to 40%.

22. The method according to claim 21, wherein the ethylenically unsaturated compound or olefin compound having one or more C═C double bonds is a C₃-C₂₀ ethylenically unsaturated compound having one or more C═C double bonds; and/or
the concentration of the hydrogen halide is from 10 to 40%.

23. The method according to claim 22, wherein the ethylenically unsaturated compound or olefin compound having one or more C═C double bonds is: ethylene, propylene, butylene, butadiene, pentene, pentadiene, hexene, hexadiene, heptene, heptadiene, octene, octadiene, decene, decadiene, nonene, nonadiene, undecene, dodecene, dodecadiene, dodecatriene, docosatriene, styrene, methyl styrene or divinyl benzene; and/or
the concentration of the hydrogen halide is from 20 to 40%.

24. The method according to claim 16, wherein the molar ratio of the ethylenically unsaturated compound or the olefin compound to the hydrogen halide in the step (1) is from 1:1.0-10.

25. The method according to claim 16, wherein the molar ratio of the ethylenically unsaturated compound or the olefin compound to H₂O₂ in the step (1) is 1:0.9-20; and/or
the concentration (wt %) of the H₂O₂ is 8-90%.

26. The method according to claim 25, wherein the molar ratio of the ethylenically unsaturated compound or the olefin compound to H₂O₂ in the step (1) is from 1:1.0-10; and/or
the concentration (wt %) of the H₂O₂ is 10-80%.

27. The method according to claim 26, wherein the molar ratio of the ethylenically unsaturated compound or the olefin compound to H₂O₂ in the step (1) is from 1:1.1-5; and/or
the concentration (wt %) of the H₂O₂ is 15-70%.

28. The method according to claim 2, wherein the molar ratio of the halohydrin to the alkali metal hydroxide in the step (2) is 1:1.0-10.

29. The method according to claim 17, wherein the reaction temperature in the step (1) is from 10 to 60° C., and/or, the reaction temperature in the step (2) is 5-100° C.

30. The method according to claim 29, wherein the reaction temperature in the step (1) is from 10 to 50° C., and/or, the reaction temperature in the step (2) is 10 to 90° C.
